(19) United States
(12) Patent Application Publication
(10) Pub. No.: US 2024/0389898 A1
(43) Pub. Date: Nov. 28, 2024

(54) ELECTROCHEMICAL SENSORS

(71) Applicant: Universal Biosensors Pty Ltd, Rowville, Victoria (AU)

(72) Inventors: George Warren Greene IV, Victoria (AU); Simon Moulton, Victoria (AU); Saimon Moraes Silva, Victoria (AU); Anita Quigley, Victoria (AU); Robert Kapsa, Victoria (AU); Daniel Peter Langley, Victoria (AU)

(21) Appl. No.: 18/686,321

(22) PCT Filed: Aug. 23, 2022
(86) PCT No.: PCT/AU2022/050964

§ 371 Date: Feb. 23, 2024

Related U.S. Application Data
(60) Provisional application No. 63/363,946, filed on Apr. 29, 2022.
(60) Provisional application No. 63/256,504, filed on Oct. 15, 2021.
(60) Provisional application No. 63/260,515, filed on Aug. 23, 2021.

(51) Int. Cl. A61B 5/145; A61B 5/00; A61B 5/1477

(52) U.S. Cl. CPC A61B 5/14546; A61B 5/1451; A61B 5/14517; A61B 5/1477; A61B 5/742; A61B 2560/0468; A61B 2562/0295

(57) ABSTRACT

The invention disclosed generally relates to sensors, systems and methods for the detection of an analyte in a complex sample.

### CROSS REFERENCE TO RELATED APPLICATIONS

This application claims priority to U.S. Provisional Application Ser. No. 63/260,515, filed Aug. 22, 2021, U.S. Provisional Application Ser. No. 63/256,504 filed Oct. 15, 2021, and U.S. Provisional Application Ser. No. 63/363,946 filed Apr. 29, 2022, each of which is hereby incorporated by reference in its entirety.

### TECHNICAL FIELD OF THE INVENTION

This invention generally relates to sensors, systems and methods for the detection of an analyte in a complex sample.

### BACKGROUND TO THE INVENTION

The potential of electrochemical sensors able to function with complex samples, such as whole blood, without the need for sample preparation is demonstrated by blood glucose meters. Nevertheless, the success of these revolutionarily devices has not yet been expanded for many other biomarkers. This is due to the issue of the nonspecific adsorption of proteins on electrode surfaces, which leads to electrode fouling. Fouling hinders electron transfer and eventually leads to sensors failure (1). As glucose is a small molecule, this issue could be solved by using semi-permeable membranes that are permeable for glucose but stop proteins from reaching the electrode (2). A significant number of clinically appropriate biomarkers are similar to in properties and size as the fouling species, that is, proteins. For detection of these markers semi-permeable membranes cannot be applied (3). One of the biggest obstacles to electrochemically detect proteins in biological fluids remains the fouling of the electrode surface. This provides the impetus for the development of sensor surfaces capable of operating in complex fluids for the detection of a broad range of biomarkers.

Affinity-based electrochemical sensors including aptamers and those using antibodies are alternatives for disposable, inexpensive, and sensitive multiplexed point-of-care diagnostics for home health care (4-6). An example of these type of sensors that have been around since early 2000's is the configuration introduced by the Plaxco group, where a redox-marker modified nucleic acid probe is tethered to a solid electrode (7-9). The binding of the nucleic acid probe to the target analyte (this could be DNA and RNA targets, proteins, inorganic ions, and even small molecules) reduces the efficiency with which the attached redox marker approaches the electrode, producing an easily measured change in electron transfer efficiency (7-11). However, even though these sensors rely on the specific affinity of the attached probe to the target species, they are still susceptible to biofouling and the associated problems (i.e. loss of sensitivity and reliability of measurements). The Plaxco approach also uses alkanethiol self-assembled monolayer as a linker to tether the nucleic acid probe to the surface as well as an intercalator, hence limiting its applicability only to gold surfaces. Finally, there are multiple steps involved in the sensor surface preparation and a long time for the target analyte hybridization may be required. Thus, there remains a need to develop sensor surfaces capable of operating in complex fluids for the detection of a broad range of biomarkers without fouling of the electrode surface.

### SUMMARY OF THE INVENTION

Embodiments of the invention disclosed herein relate to electrochemical sensor for determining the presence of an analyte in a liquid sample. In some embodiments, the sensor includes an electrode having a surface, an active polymer brush, an analyte recognition element bound to the polymer brush, and a signal transduction element comprising a reporter, wherein the analyte recognition element specifically interacts with an analyte, when present, resulting in a detectable change in a charge transfer between the reporter and the electrode.

In some embodiments, the electrode is carbon-based. In some embodiments, the electrode is metal-based. In some embodiments, the electrode is conductive metal oxide-based. In some embodiments, the electrode is conductive polymer-based.

In some embodiments, the polymer brush has anti-fouling properties. In some embodiments, the polymer brush is tethered to the electrode surface. In some embodiments, the polymer brush is end-tethered to the electrode surface. In some embodiments, the polymer brush is a telechelic polymer tethered at each end to the electrode surface. In some embodiments, the polymer brush comprises a synthetic polymer. In some embodiments, the polymer brush comprises a biopolymer. In some embodiments, the polymer brush comprises lubricin.

In some embodiments, the reporter is a redox reporter. In some embodiments, the reporter is an electro-chemiluminescent species.

In some embodiments, the analyte recognition element is a protein. In some embodiments, the analyte recognition element is an antibody. In some embodiments, the analyte recognition element is a lectin. In some embodiments, the analyte recognition element is a nucleic acid. In some embodiments, the analyte recognition element is an aptamer.

In some embodiments, the liquid sample is selected from blood, serum, saliva, urine, sweat, interstitial fluid, spinal fluid, cerebral fluid, tissue exudates, macerated tissue samples, cell solutions, intracellular compartments, water, food, groundwater, or other biological and environmental samples. In some embodiments, the liquid sample is a complex liquid sample. In some embodiments, the complex liquid sample requires minimal or no processing prior to use. In some embodiments, the liquid sample is whole blood.

In some embodiments, the change in a charge transfer between the reporter and the electrode is detected using techniques selected from voltametric, pulsed, amperometric, potentiometric, impedance spectroscopy, galvanostatic impedance spectroscopy, or mixed mode. In some embodiments, the voltametric techniques are selected from linear sweep voltammetry, cyclic voltammetry, fast cyclic voltammetry and AC voltammetry. In some embodiments, the pulsed techniques are selected from differential pulse voltammetry, square wave voltammetry, and normal pulse voltammetry. In some embodiments, the amperometric techniques are selected from chronoamperometry, multistep amperometry, fast amperometry, pulsed amperometric detection, and multiple pulse amperometry. In some embodiments, the potentiometric techniques are selected from open circuit potentiometry, chronopotentiometry, linear sweep potentiometry, multistep potentiometry, and stripping chronopotentiometry.

In some embodiments, an electrochemical sensor for determining the presence of an analyte in a liquid sample is disclosed. In some embodiments, the electrochemical sensor includes an electrode, a semi-permeable, antifouling polymer brush, an analyte recognition element bound to the electrode and residing wholly within the polymer brush, and a signal transduction element comprising a reporter, wherein the analyte recognition element specifically interacts with an analyte, when present, resulting in a detectable change in a charge transfer between the reporter and the electrode.

In some embodiments, the electrode is carbon-based. In some embodiments, the electrode is metal-based. In some embodiments, the electrode is conductive metal oxide-based. In some embodiments, the electrode is conductive polymer-based.

In some embodiments, the polymer brush has anti-fouling properties. In some embodiments, the polymer brush is tethered to the electrode surface. In some embodiments, the polymer brush is end-tethered to the electrode surface. In some embodiments, the polymer brush is a telechelic polymer tethered at each end to the electrode surface. In some embodiments, the polymer brush comprises a synthetic polymer. In some embodiments, the polymer brush comprises a biopolymer. In some embodiments, the polymer brush comprises lubricin.

In some embodiments, the reporter is a redox reporter. In some embodiments, the reporter is an electro-chemiluminescent species.

In some embodiments, the analyte recognition element is a protein. In some embodiments, the analyte recognition element is an antibody. In some embodiments, the analyte recognition element is a lectin. In some embodiments, the analyte recognition element is a nucleic acid. In some embodiments, the analyte recognition element is an aptamer.

In some embodiments, the liquid sample is selected from blood, serum, saliva, urine, sweat, interstitial fluid, spinal fluid, cerebral fluid, tissue exudates, macerated tissue samples, cell solutions, intracellular compartments, water, food, groundwater, or other biological and environmental samples. In some embodiments, the liquid sample is a complex liquid sample. In some embodiments, the complex liquid sample requires minimal or no processing prior to use. In some embodiments, the liquid sample is whole blood.

In some embodiments, the change in a charge transfer between the reporter and the electrode is detected using techniques selected from voltametric, pulsed, amperometric, potentiometric, impedance spectroscopy, galvanostatic impedance spectroscopy, or mixed mode. In some embodiments, the voltametric techniques are selected from linear sweep voltammetry, cyclic voltammetry, fast cyclic voltammetry and AC voltammetry. In some embodiments, the pulsed techniques are selected from differential pulse voltammetry, square wave voltammetry, and normal pulse voltammetry. In some embodiments, the amperometric techniques are selected from chronoamperometry, multistep amperometry, fast amperometry, pulsed amperometric detection, and multiple pulse amperometry. In some embodiments, the potentiometric techniques are selected from open circuit potentiometry, chronopotentiometry, linear sweep potentiometry, multistep potentiometry, and stripping chronopotentiometry.

In some embodiments, methods for determining the presence of an analyte in a liquid sample using the electrochemical sensors described herein are disclosed. In some embodiments, the methods include exposing the analyte recognition element to analyte species present in a sample, under condition sufficient to permit binding of the recognition element and making at least one electrical measurement indicating the change in charge transfer due to the interaction of the reporter with the working electrode to detect the presence, concentration and/or amount of the analyte species in the sample.

In some embodiments, methods for determining the presence of a target analyte in a liquid sample are disclosed. In some embodiments, the methods include exposing the analyte recognition element of the sensor of claim 1 to a liquid sample suspected of containing a target analyte, under condition sufficient to permit binding of the target analyte to the recognition element; and performing at least one electrical measurement to detect a change in charge transfer due to the interaction of the reporter with the working electrode, wherein the change in charge transfer indicates the presence, concentration and/or amount of the target analyte in the liquid sample. In some embodiments, the electrical measurement results in an electrochemiluminescence response that correlates with the concentration of the target analyte in the liquid sample.

In some embodiments, methods of making the electrochemical sensors described herein are disclosed. In some embodiments, the methods include depositing an active polymer brush onto a conductive electrode surface by self-assembly; and tethering an analyte recognition element to the active polymer brush. In some embodiments, the methods include tethering an analyte recognition element to a conductive electrode surface; and depositing an active polymer brush onto the electrode surface to embed the analyte recognition element.

In some embodiments, a sensor device including at least two electrodes each having an active surface that interact with a liquid sample are disclosed. In some embodiments, a first electrode contains a sensor disclosed herein and a second electrode provides a current and reference potential. In some embodiments, the first and second electrode active surfaces are opposing and separated by a non-conductive spacer that forms a channel into which the liquid sample can be introduced. In some embodiments, the first and second electrode active surfaces are co-planar.

In some embodiments a sensor device including at least three electrodes is disclosed. In some embodiments, the sensor device includes a combination of opposing and co-planar arrangement. In some embodiments, the sensor device includes at least two substrate materials coated with conductive material, wherein at least one of the two substrate materials coated with conductive material is patterned by printing, mechanical punching or laser scoring/ablation to isolate at least two separate electrodes thereon, wherein at least one electrode has active chemistry deposited on it to provide sensitivity and selectivity to an analyte of interest; and a nonconductive spacer material used to separate the at least two substrate materials coated with conductive material and to define a fixed volume within the sensor device.

In some embodiments, methods of manufacturing the sensor devices disclosed herein are disclosed. In some embodiments, the methods of manufacturing are by batch and continuous processing. In some embodiments, the methods of manufacturing include depositing a self-assembled active polymer brush onto a first conductive surface layer; dispensing an analyte recognition element labelled with a reporter onto the polymer brush; patterning the first conductive surface to isolate at least two electrodes; introducing a nonconductive spacer layer to define an electrode active volume on the first conductive surface layer; providing a second conductive surface layer; and assembling the first and second conductive surface layers into an opposing laminate construction. In some embodiments, the second conductive surface layer is patterned.

In some embodiments, a method of making a device comprising at least two electrodes as disclosed herein are disclosed. In some embodiments, the device comprising at least two electrodes includes a first electrode contains a sensor disclosed herein and a second electrode provides a current and reference potential. In some embodiments, the first and second electrodes have active surfaces and the first electrode active surface and the second electrode active surface are opposing and separated by a non-conductive spacer that forms a channel into which a liquid sample can be introduced. In some embodiments, the first and second electrodes have active surfaces and the first electrode active surface and the second electrode active surface are co-planar. In some embodiments, the device comprises three electrodes in a combination of opposing and co-planar arrangements.

### BRIEF DESCRIPTION OF THE DRAWINGS

FIG. 1 is a schematic illustration of the steps involved in the sensing approach for detection of Tn antigen in whole blood. The sensor interface includes a redox-reporter-modified peanut agglutinin that works as a recognition/receptor element attached to a disposable reduced graphene oxide electrode via a lubricin self-assembled monolayer on the graphene oxide electrode. Signal generation happens when a target antigen, in this case, a Tn antigen, binds to the recognition element, diminishing the efficacy with which the attached redox-reporter, methylene blue, transfer electrons to the electrode surface as measured using square wave voltammetry as excitation technique.

FIG. 2 depicts surface characterization of the single-step reagentless protein-based sensor. FIG. 2a shows a representative QCM result showing the adsorption of lubricin on to a gold electrode surface followed by the binding of PNA to the adsorbed lubricin layer. FIG. 2b depicts cyclic voltammogram of a bare rGO disposable electrode (solid line), rGO electrode modified with lubricin only (dashed line), rGO electrode modified with lubricin and PNA without the redox-reporter (dotted line), and rGO electrode modified with lubricin and MB-PNA (dashed-dotted line). FIG. 2c shows a plot of different scan rates versus anodic and cathodic peak currents for the reduced graphene oxide electrode coated with a lubricin brush containing bound MB-PNA. FIG. 2d shows representative square-wave voltammograms (frequency 25 Hz, pulse amplitude 25 mV).

FIG. 3 depicts analysis of the sensitivity of the electrochemical measurement of the concentration of Tn antigen. FIG. 3a shows representative square-wave voltammograms (frequency 25 Hz, pulse amplitude 25 mV) obtained before (solid line) and after (dashed line) spiking 5.15×10⁻⁹ M Tn antigen solution in to blood samples from a healthy donor. FIG. 2b shows binding-induced change in the square wave current after exposure of the sensor to different concentrations of Tn antigen, obtained by serial dilution of a stock solution of Tn antigen spiked in PBS (squares) and healthy human whole blood (circles). Each point is the mean of square wave peak currents of three independent electrodes±standard errors (σ/√n).

FIG. 4 depicts analysis of Tn antigen in samples extracted from different human cancer cell lines and human whole blood sample with breast cancer. Binding-induced change in current after the sensor exposure to liver metastasis cell culture media (FIG. 4a), Merkel cell carcinoma cell culture media (FIG. 4b), and pancreatic cancer cell culture media (FIG. 4c). FIG. 4d shows representative square-wave voltammograms (frequency 25 Hz, pulse amplitude 20 mV) obtained in blood samples from a healthy donor (solid line) and whole blood sample for a breast cancer donor (dot-dashed line). Background peak currents were subtracted from the square-wave voltammograms to give the presented results. FIGS. 4e and 4f show reproducibility of the square wave current through ten cycles of square wave voltammetry (frequency 25 Hz, pulse amplitude 25 mV) of the prepared sensor after exposure to whole blood from a healthy donor and whole blood from breast cancer donor respectively. Each point shows the average of data points from 3 different prepared sensors. Error bars represent the standard errors.

FIG. 5 depicts a mechanism that can be used in an electrochemiluminescence modified platform. The active polymer brush can be adapted to perform electrochemiluminescence using the mechanism. In this case, the analyte recognition element (e.g., aptamer or single nucleic acid strand) is modified with a redox reporter which is an electrochemiluminescence probe. Upon oxidation/reduction of this probe, a chromophore/luminophore/fluorophore becomes activated causing the emission (or adsorption) of light which can be detected and quantified. The intensity of the light (or adsorption) is then used to quantify the amount of bound analyte.

FIG. 6 shows a schematic representation of a disposable three-electrode strip according to one of the embodiments of the invention disclosed herein. In this embodiment, gold is used as the working electrode, gold as counter and palladium as reference electrode. For sample analysis, the biological fluid can be easily introduced into the channel as indicated in the scheme. An insulating spacer is used to define the geometric area of the working, counter and reference electrodes.

FIG. 7 shows the proposed mechanism of the binding between PFAS and human liver fatty acid-binding protein. Figure reproduced from Environ. Sci. Technol. 2020, 54, 5676-5686

FIG. 8a shows a representative square-wave voltammogram for a reduced graphene oxide disposable electrode modified with lubricin and fatty-acid binding protein from liver functionalized with methylene blue recorded in the absence (solid line) and presence of different increasing concentrations of PFOA (dashed lines). Experiments were recorded using 5 Hz. FIG. 5b depicts a calibration curve for different concentrations of PFOA (96 nM, 966 nM, 9.6 μM, 48 μM, 96 μM, 966 μM). Error bars represent the standard deviation for three measurements using three independent electrodes.

FIG. 9 shows a schematic representation of an exemplary double interpenetrating brush sensor surface. In this embodiment of the active polymer brush, the analyte recognition element is tethered directly to the electrode surface forming a small polymer brush embedded within the active polymer brush which prevents electrode fouling and supports the smaller brush reducing electrode dynamics.

FIG. 10 shows a double interpenetrating brush sensor response in both cyclic voltammogram (FIG. 10a) and square-wave voltammogram (FIG. 10b) for the detection of miRNA-375 using the nucleic acid lubricin embedded sensor. Experiments were carried out in whole blood (solid line) and whole blood spiked with 0.3 μM miRNA-375 (dashed line).

FIG. 11 shows an exemplar embodiment of a sensor device for multiplexed detection, comprised of three working electrodes and a reference electrode defined by patterning a conductive material on a nonconductive substrate. A patterned spacer is placed on top of this layer and a counter electrode is then placed opposing the other electrodes.

FIG. 12 shows another embodiment of a sensor device for multiplexed detection in which two substrate materials are coated with conductive materials. Both conductive layers are patterned such that the counter and reference electrodes are positioned opposing three working electrodes.

### DETAILS OF INVENTION

The invention disclosed herein relates to sensors for the detection of an analyte in a complex sample. The basic components of the sensor include an electrode, a polymer brush, a recognition element, and a signal transduction element. The components can be incorporated into sensor assemblies or sensor systems for the detection of target species by various electrochemical interrogation techniques. The various elements of the invention are described in detail below.

The sensing platform disclosed herein offers a fast, easy and precise way of creating an ultrasensitive point-of-care tumor-associated antigen biosensor for direct analysis in patient samples. This assay presents a variety of benefits over existing methods used to quantify Tn antigen (fluorescence polarization assays and HPLC). The analysis time can be as short as few seconds, the assay requires relatively inexpensive supporting electronics, is compatible with automation, and is capable of multiplexing strategies both via spatial separation of reporting chemistries to separate electrodes or using a single electrode pair with different reporters.

### Analytes

The sensing elements of the invention disclosed herein are directed to the detection of a target species. The target species can include any inorganic or organic molecule, for example, but not limited to: a tumor antigen, a small molecule drug, a metabolite, a hormone, a peptide, a protein, a carbohydrate, a nucleic acid, a lipid, a hormone, a metabolite, a growth factor, a neurotransmitter, a nutrient, a pollutant, pathogen, pathogen-induced or pathogen-derived factor, or any other composition of matter. In addition to molecules, the target species can include, for example, large and complex targets, such as a cell.

### Electrodes

In embodiments of the invention, the electrode used herein can include any suitable electrode material for electrochemical sensing, including, but not limited to, carbon, titanium, tungsten, platinum, aluminum, copper, palladium, mercury films, silver, oxide-coated metals, semiconductors, graphite, carbon nanotubes, gold and any other conductive material upon which biomolecules can be conjugated. In some embodiments, the sensor includes a carbon-based screen-printed electrode.

The electrode can be configured in any desired shape or size, including discs, strips, rectangular electrodes, electrode arrays, wires, and other configurations.

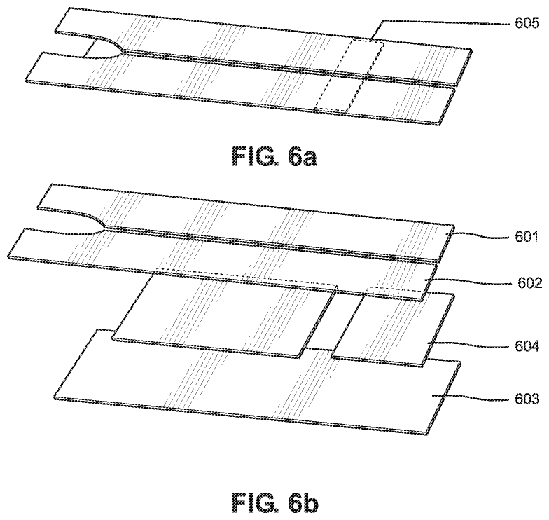

In some embodiments, disposable test strips disclosed herein comprise two electrodes in a co-planar or opposing arrangement. In some embodiments, disposable test strips comprise a three or more electrode arrangements such as the exemplary arrangements shown in FIGS. 6, 11 and 12.

In some embodiments disclosed herein, the detection of any analyte found in biological fluids or other sample solutions relies on the interactions between analyte and the interface of the working electrode at the test strip where monitoring both potential and current is performed simultaneously. In some embodiments, this is achieved using a three-electrode electrochemical cell, where the analyte/electrolyte is investigated at the interface with the so-called “working electrode.” In some embodiments, the system is perturbed (i.e., taken away from its equilibrium), for example, by polarizing the electrode either cathodically or anodically by applying a potential difference between the working electrode and a second electrode called the counter electrode. In some embodiments, the counter electrode completes the circuit by providing a source of current into the electrolyte to balance the electrochemical reactions happening at the working electrode. In some embodiments, to apply a known potential, a third electrode is used as a standard/reference electrode. The third electrode acts as a reference by allowing the measuring and control of the potential between the counter and working electrodes without passing any current. After perturbation, the effect of perturbation is recorded. For example, in some embodiments, the current that passed between the working and the counter electrode is recorded. In some embodiments the optical emission of a reporter in response to the perturbation is recorded. In some embodiments, using a three-electrode configuration allows the use of potentiometric techniques and electrochemical transient techniques, such as, but not limited to, differential pulse voltammetry and square-wave voltammetry, or any other electrochemical technique.

FIG. 6 shows a schematic representation of an exemplary three-electrode strip disclosed herein. In some embodiments of the three-electrode strip disclosed herein, gold is used as the working electrode (601). In some embodiments, gold is used as the counter electrode (602). In some embodiments, palladium is used as the reference electrode (603). In some embodiments, an insulating spacer (604) is used to define the geometric area of the working (601), counter (602), and reference (603) electrodes. Additionally the working (601) and counter (602) electrodes are defined by patterning the electrode surface, either by laser ablation, mechanical punching or during deposition of the conductive material, and the like. In some embodiments, the reference electrode (603) is opposing the working electrode (601), which is quite different from the co-planar disposable electrodes available currently in the market. In some embodiments, the reference electrode (603) is opposing the working electrode (601) and palladium is used as a reference electrode (603). In some embodiments, the reference electrode (603) is opposing the working electrode (601) and gold is used as the working electrode (601) and counter electrode (602), and palladium is used as a reference electrode (603). In some embodiments, the strips are produced using gold webs and palladium webs. In some embodiments, the positions of the reference electrode and counter electrode is reversed. In some embodiments, the materials used for any of the electrodes is replaced with other conductive materials.

For sample analysis, the sample solution can be easily introduced into the channel defined by the spacer material and electrode surfaces (605). In some embodiments, only a few microliters of a sample for analysis are required to completely fill the strip channel (605).

### Recognition Elements

The recognition element used in embodiments disclosed herein can include any method of binding an analyte or target known in the art. This can include, for example, but not limited to, a DNA aptamer, RNA aptamer, an aptamer including non-natural nucleic acids, proteins, antibodies, lectins, lectin binding domains, synthetic peptides, molecularly imprinted polymers, and the like, as well as hybrids of the foregoing. Variants wherein the recognition element is a nucleic acid other than an aptamer, or is other than a nucleic acid, for example sensors using small molecules or fragments of proteins or antibodies, are also within the scope of the invention.

In some embodiments, the recognition element is a lectin. For example, in some embodiments, peanut agglutinin (PNA) is used to detect Tn antigen.

### Signal Transduction Elements

In some embodiments, the sensors disclosed herein include a signal transduction element also referred to as a “reporter”. In some embodiments, the signal transduction element includes, for example, but not limited to, electrochemically active species, redox reporters, fluorescent markers, (e.g., FITC, quantum dots, carbon nanodots, GFP etc.) electro-chemiluminescent markers (e.g., Tris(bipyridine) ruthenium (II) chloride, etc.), and the like. In some embodiments, the recognition element is modified with a reporter. The reporter can take the form of any active molecule or particle which will provide a measurable response upon the application of a stimulus. This includes, for example, but is not limited to, electrochemically active species (redox reporters), lumiphores (chemiluminescent, fluorescent, electro-chemiluminescent), chromophores (chemical species which change colour as a result of the analyte presence), and the like.

Redox reporters are chemical entities capable of electron transfer to or from the electrode in response to an excitation stimulus, for example, a potential step applied to the working electrode in a voltametric scan. With sufficient proximity and accessibility of a redox reporter to the electrode, an electrical signal, e.g., current, voltage, or other measurable electrical interaction, will occur between the redox reporter and the electrode upon excitation of the electrode.

In some embodiments, the redox reporter is positioned on the recognition element as in conventional electrochemical sensors, wherein binding of the target species to the recognition element causes conformational change in the recognition element which changes the accessibility of the sensing redox reporter with respect to the electrode. In some embodiments, steric-interference effects from target binding cause measurable change in the electrical signal generated by the sensing reporter. In some embodiments, binding causes the sensing reporter to be excluded from a pocket on the recognition element, improving its electron transfer abilities. Recognition elements can be configured for turn-off sensing, turn-on sensing, or strand displacement sensing, as known in the art. The placement of such sensing reporters can be at the 5′ end, 3′ end, or otherwise engineered using known methods.

Exemplary redox species include, but are not limited to, methylene blue, ferrocene, viologen, anthraquinone or any other quinones, daunomycin, organo-metallic redox reporters, for example porphyrin complexes or crown ether cycles or linear ethers, ruthenium, bis-pyridine, tris-pyridine, bis-imidizole, cytochrome c, plastocyanin, ethylenetetracetic acid, and the like.

### Polymer Brush

In some embodiments, recognition elements are bound to the surface of an electrode by an anchoring moiety. In some embodiments, the anchoring moiety is a polymer brush. In some embodiments, the anchoring moiety includes elements that form self-assembled monolayers on the electrode surface. In some embodiments, the anchoring moiety is not covalently linked to the electrode. In some embodiments, the recognition elements are deposited on the electrode surface at any desired density, for example, in the range of 1×10⁹ to 1×10¹² molecules/cm² and then incorporated into the polymer brush.

In some embodiments, the polymer brush is lubricin (LUB). Lubricin is a glycoprotein commonly found in synovial fluids and covering the cartilage surface of mammals. In some embodiments, lubricin is used in the sensor as a linker to anchor the site-specific receptor as well as antifouling agent (12,13). In some embodiments, lubricin forms a self-assembled ‘telechelic’ polymer brush grafted onto an electrode surface. During self-assembly, the lubricin molecule adheres to the electrode surface through globular end-domains located at the C- and N-terminus of the protein via a combination of electrostatic, hydrophobic, and/or Van der Waals interactions. The terminal end-grafting of the lubricin molecule leads the central and highly glycosylated mucin domain region of the protein to form a loop confirmation which presents this domain to the solution. Close packing of lubricin molecule loops results in the extension of mucin domain loops and the adoption of the polymer brush architecture. The thickness of the mucin domain loop is roughly ½ the ‘contour’ length of the central mucin domain (˜90-130 nm) and exhibits a ‘smooth’ surface with an RMS roughness which is typically equivalent to that of the electrode surface on which it is coated; e.g., <1 nm on mica.

The lubricin molecule is heavily glycosylated, the glycan profile of recombinant lubricin consists almost entirely of core-1 O-linked glycans. Glycome analysis has reported 172 glycans (34). The heavy glycosylation of the lubricin molecule produces many sites for the attachment of lectins specific to individual glycans which can be found along the mucin domain. Although not comprehensive, lectins that can bind to the mucin domain include, but are not limited to:
1) ABL—Agaracus Bisporus Lectin (White Button Mushroom)
2) PNA—Peanut agglutinin
3) Bauhinia Purpurea Lectin (BPL)
4) Solanum Tuberosum (Potato) Lectin (STL, PL)
5) Vicia Villosa Lectin (VVL, VVA)
6) Wisteria Floribunda Lectin (WFA, WFL)
7) Wheat Germ Agglutinin (WGA)
8) Galectin-3

In some embodiments, lectins and their binding domains provide a mechanism for conjugating the strongly anti-adhesive mucin domain with analyte recognition elements.

In some embodiments, attachment of non-lectin based analyte recognition elements is achieved by the grafting or conjugation of lectins with the recognition elements or via molecular engineering approaches where specific lectin binding domains or recognition elements are introduced during the recombinant or synthetic synthesis of peptides, proteins, enzymes and antibodies. Bi-valent aptamers, where one side binds to LUB while the opposite side binds an analyte, can also be used as an analyte recognition element.

In some embodiments, lubricin is used as antifouling coating while still preserving the electrochemical properties of the underlying electrode surface (13,15,16). Lubricin self-assembles into a telechelic brush. This brush is unique because it is fully extended even at relatively low grafting density (˜9 nm between end-domains). The diffuse architecture of the lubricin brush (it is >95% water) enables the flexibility for the sensor and is also responsible for preserving the electrochemical activity of the electrode.

In some embodiments, the polymer brush acts to prevent fouling of the electrode surface providing a stable electron transfer rate to and from the transduction element. The brush also provides a functional chemistry to attach analyte recognition elements and signal transduction elements to the electrode surface. The main advantage of this approach is that the Lubricin end domains adhere non-specifically with high affinity to a wide variety of surfaces.

### Electronic Components

In some embodiments, the sensor assemblies further include or are in connection with appropriate electronic components for performing electrochemical measurements. The electronic components can include, for example, two or more devices in electrical and/or network connection with one another, or can include a single integrated device.

In some embodiments, electronic components include potentiostats or other voltage sources and voltage controllers. In some embodiments, the system further includes appropriate circuitry for reading sensor outputs, and storing such outputs or routing the outputs to other devices. In some embodiments, the systems further include data processing means, for example, a general-purpose computer or other data processor capable of carrying out the various calculations utilized in the methods of embodiments of the invention disclosed herein. In some embodiments, the system further includes non-transitory computer-readable recording media having stored thereon an encoding program that causes a computer to execute a process, the process comprising one or more data processing calculations for readout and interpretation of signals from a sensing element.

### Methods

In some embodiments, the invention provides methods of measuring the concentration of a target species in a sample using the electrochemical sensors disclosed herein.

In some embodiments, the invention provides methods of measuring the concentration of a target species in a sample using the optical readout methods described herein.

In some embodiments, the methods disclosed herein are carried out by exposing the sensing element to a sample. The sample can include a liquid. The sample can include, for example, but is not limited to, whole blood, serum, saliva, urine, sweat, interstitial fluid, spinal fluid, cerebral fluid, tissue exudates, macerated tissue samples, cell solutions, intracellular compartments, water, food, groundwater, or other biological and environmental samples. In some embodiments, the sample is derived from a subject, for example a human patient or a non-human animal such as a veterinary subject or test animal. In some embodiments, the sample is finger-prick volume of unprocessed or processed blood. In some embodiments, the sample is processed, such as by filtering, dilution, buffering and the application of other materials or processes to the sample prior to analysis.

In some embodiments, measurement is made over a selected time interval when the sensing element of the electrochemical sensor is contacted with the sample. In some embodiments, measurements over the time interval are made by voltametric interrogation of the electrode to assess sensor response (either electrical or optical) at specific voltages. Interrogation can be by any voltametric method, including, but not limited to, cyclic voltammetry, differential pulse voltammetry, alternating current voltammetry, square wave voltammetry, potentiometry or amperometry, impedance spectroscopy, and other similar methods known in the art. Waveforms can be selected as known in the art. Each scan will be performed across a range of voltages which results in distinct signals being generated from the sensing redox reporter and reference redox reporter. In some embodiments, scans are performed at discreet time intervals or continuously over the measurement period.

In embodiments including lubricin, lectin and a redox-reporter, the underlying transduction mechanism of the sensor system is centered on the change in molecular dynamics resulting from the binding of an analyte to the lubricin/lectin/redox reporter construct. For example, in some embodiments, in the absence of the analyte, the lubricin molecule bends and coils with an average frequency and, as it does, it brings the tethered lectin/redox reporter into close proximity to the electrode interface for a given fraction of time. While the redox reporter is near the electrode, it can participate in oxidation/reduction reactions and so will generate a current (in square wave voltammetry) which is proportional to the fraction of time it is in proximity; i.e., the bending/coiling frequency. In some embodiments, the binding of the analyte (e.g., Tn) alters the molecular dynamics of individual lubricin/lectin/redox reporter complexes which thus shifts the average bending/coiling frequency proportionately to the number of complexes whose dynamics are altered by analyte binding. The shifting average frequency therefore changes the average time fraction the redox reporter is near the electrode resulting in an altered current response in square wave voltammetry. The current response can either increase or decrease with analyte binding depending upon the specific impact the analyte has on the collective dynamics of the lubricin brush system or if the chosen frequency of the measurement is primarily sampling the analyte-bound (increasing signal) or unbound (decreasing signal) populations.

In some embodiments, an optical reporter is included, and the sensor electrode is excited by incident light as known in the art. As a result of the collective dynamics of the lubricin brush system the reporter will only fluoresce in the presence of the analyte.

### Method of Making the Sensors

In some embodiments, methods of making the sensors are also provided. In some embodiments, the methods include covalently labelling a reporter to a recognition element. In some embodiments, sequential deposition is used. In some embodiments, the electrode surface is coated with a self-assembling polymer brush followed by the conjugation of the recognition element/reporter. In some embodiments, the conjugation of polymer brush with the recognition element/redox reporter is accomplished in solution (by simple mixing) followed by the self-assembly of the polymer brush/recognition element/redox reporter in a single deposition step. In some embodiments the electrode surface may be coated with an analyte recognition element covalently bonded to a reporter which is then embedded within a polymer brush forming an interdigitated polymer brush.

### Devices

The sensor surfaces described herein can be used to manufacture devices according to the invention described herein. In some embodiments, the devices disclosed herein include two electrodes, in some embodiments, these devices disclosed herein include three or more electrodes. In some embodiments, the devices disclosed herein include a working electrode functionalized with the sensor described herein. In some embodiments, the working electrode is functionalized in a separate manner.

In some embodiments, the device includes a working electrode which provides sensitivity and selectivity to an analyte of interest, and at least one additional electrode which allows the application of a current or voltage source to stimulate a response from the working electrode. In some embodiments, the device includes additional electrodes to provide a reference potential. In some embodiments, the device includes additional working electrodes for the detection of multiple analytes. In some embodiments, the device includes additional electrodes for determination of a reference potential and simultaneously additional working electrodes for multiple analytes. In some embodiments the device includes a counter electrode, reference electrode and a single working electrode functionalized with multiple analyte recognition elements labelled with different reporters to detect multiple analytes.

In some embodiments, the devices are constructed with at least two conductive surfaces separated by a nonconductive spacer material. In some embodiments, one or both conductive surfaces are patterned either by mechanical punching, screen printing, contact printing, inkjet printing or laser scoring/ablation, and the like, to define additional electrodes. In some embodiments, at least one of the electrodes is functionalized with a sensor chemistry, and at least one electrode is used to provide electrical stimulus (current or voltage) or measure a response to a chemical reaction. In some embodiments, additional electrodes are used to measure a reference potential or provide capability for additional analyte detection.

In some embodiments, a spacer material is patterned to define a region for interaction of the sample with the electrodes. The patterning can be performed by either mechanical punching, laser ablation, screen printing, contact printing or casting of the spacer material, or other similar methods known in the art. In some embodiments, the spacer material is non-conductive.

In some embodiments, the devices disclosed herein are assembled into a laminate structure wherein materials carrying the electrodes are adhered to a spacer material either by thermal adhesives, thermal bonding of the spacer material, chemical, optical or pressure sensitive adhesives, and the like.

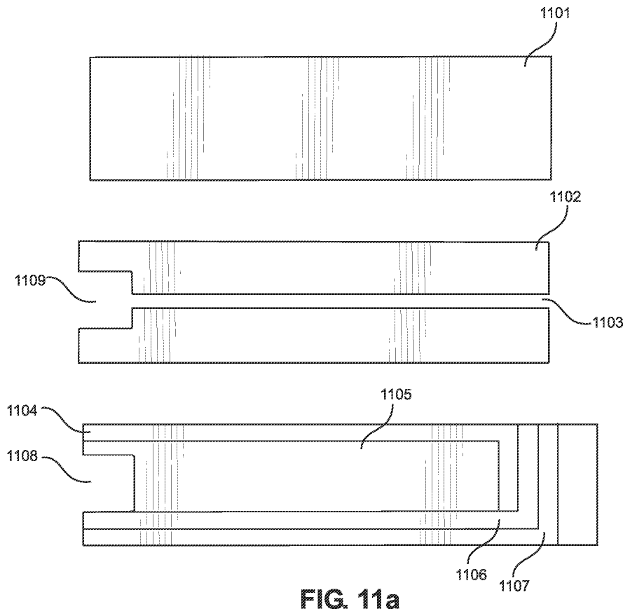

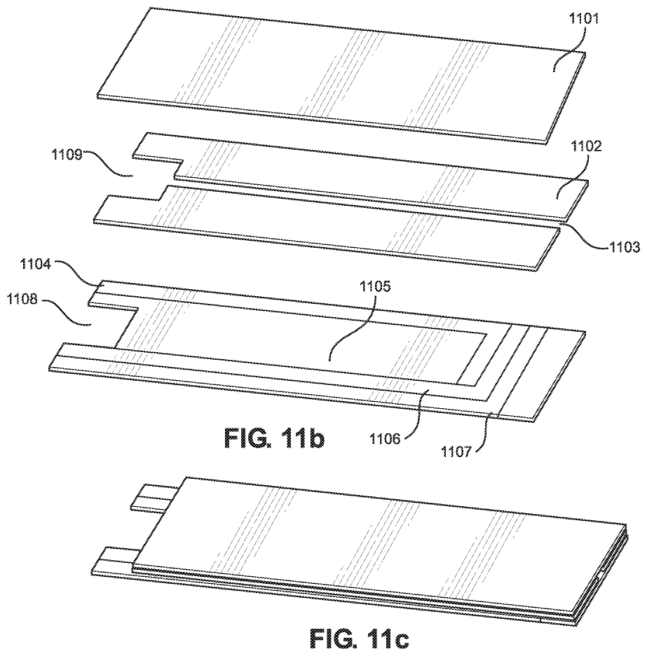

FIG. 11 shows an embodiment of a sensor device wherein the device includes a counter electrode (1101), a nonconductive spacer (1102) which has been physically patterned (1103) and (1109) to define a sample interaction area (1103) and allow electrical contact to be formed from beneath the device (1109). The lower substrate includes three working electrodes (1104), (1106), (1107) and a reference electrode (1105) defined by laser scribing. The device is designed such that counter electrode (1101) opposes the other electrodes and is separated from them by the patterned nonconductive spacer layer (1102). Both the spacer layer (1102) and the primary substrate have holes punched in them (1109) and (1108), respectively, to allow electrical connection to be made from below the device to the counter electrode (1101). In some embodiments, only one of the two conductive layers on the substrate material is patterned. In some embodiments, the active areas of the electrodes are defined by the combination of the surface patterning and spacer layers. In some embodiments, only the narrow regions of the electrodes (1104), (1105), (1106) and (1107) exposed by the sample channel (1103) contribute to the sensor response. In some embodiments, chemistry is deposited on the working electrodes (1104), (1106), (1107) in a continuous stripe perpendicular to the sample channel (1103) either three independent chemistries can be deposited (one stripe per electrode) to detect different analytes, or a single chemistry can be deposited over the three working electrodes to allow multiple measurements of the same analyte in the same sample for redundancy. The electrodes can take a range of geometries including, but not limited to, squares, circles, rings and interdigitated electrodes. FIG. 11 is merely an exemplar of one possible geometry.

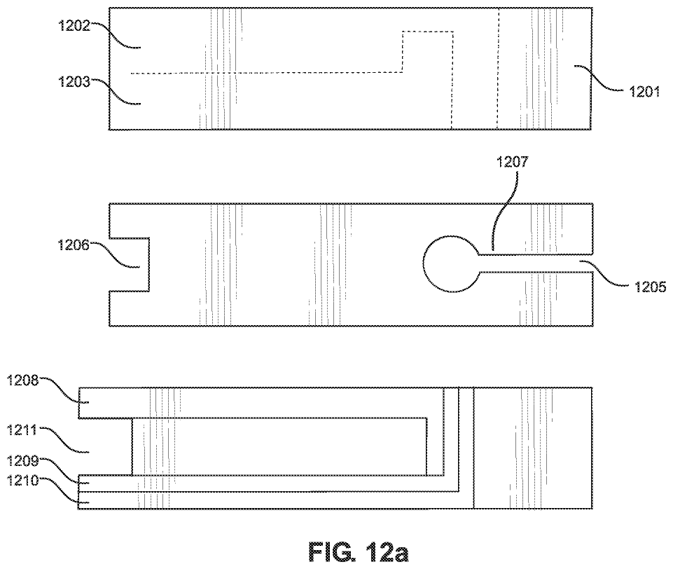

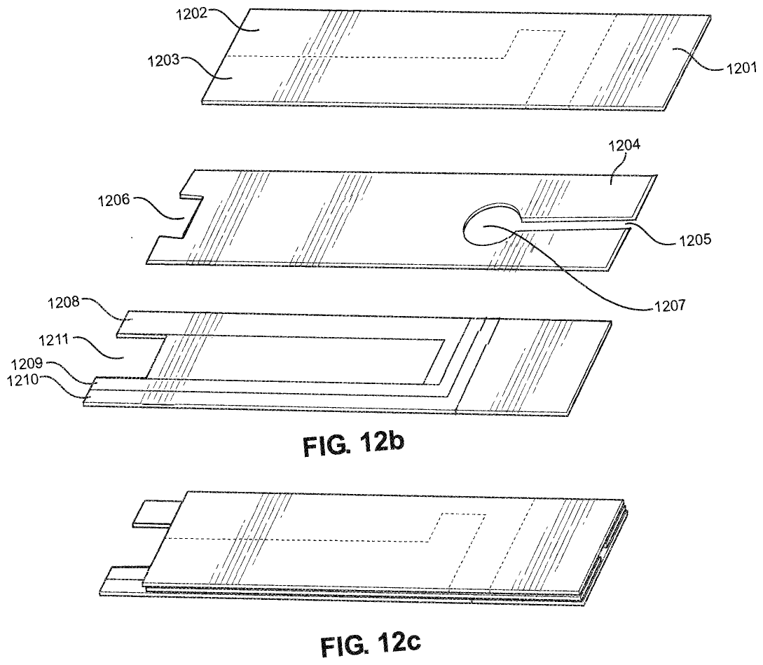

FIG. 12 shows another embodiment of a sensor device with both opposing conductive layers patterned. The dashed lines on the top layer (1201) indicate that the patterning is on the lower face of the layer. A reference electrode (1202) and a counter electrode (1203) are defined on the top layer of the device. The nonconductive spacer layer (1204) is patterned to define a sample interaction volume (1207) and provide a path for filling the device (1205). In some embodiments, vents are also included to allow air to escape as the device fills (not drawn). The space is also shaped to allow access from below to the top layer (1201) for electrical connection to the reference electrode (1202) and counter electrode (1203). The lower layer is coated with a conductive material which is patterned with three working electrodes, (1208), (1209) and (1210). Both the spacer layer (1204) and the lower layerare punched (1206) and (1211), respectively, to provide access for electrical connections to the reference and counter.

### Methods of Manufacturing the Devices

The devices disclosed herein can be manufactured in either a batch process or a continuous process (reel to reel).

In some embodiments, a substrate material (e.g., polymer, cloth or paper, and the like) is coated with a conductive material (e.g., carbon, conductive polymer, metal, conductive oxides or conductive nanoparticles, and the like), either by printing (e.g., inkjet, screen, contact), sputter coating, dipping or spray deposition, and the like. In some embodiments, a printing method is used and patterns are defined directly during the deposition. In some embodiments, other processes produce a conformal continuous coating which is patterned later. In some embodiments, substrate materials are purchased precoated with a conductive material.

In some embodiments, the electrode geometry is patterned (e.g., by laser scoring/ablation or mechanical punching, and the like) into the conductive material defining separate electrodes on the same substrate, then the sensor chemistry is deposited on the conductive material. In some embodiments, the sensor chemistry is deposited on the conductive material and the electrodes are patterned (e.g., by laser scoring/ablation or mechanical punching, and the like) after sensor chemistry deposition.

In some embodiments, a spacer material is adhered to the conductive material defining a region of the conductive material that is exposed, providing an area of interaction. In some embodiments, the spacer material is a pre-patterned nonconductive spacer material (e.g., mechanical punch or laser patterning) or a direct printing of spacer material onto the electrode. In some embodiments, the spacer material is adhered through thermal, mechanical, chemical, or optical activation.

In some embodiments, a second layer of substrate material (e.g., polymer, cloth or paper, and the like) also coated with conductive material (e.g., carbon, conductive polymer, metal, conductive oxides or conductive nanoparticles, and the like) is placed on the spacer material opposite to the first layer of conductive material. In some embodiments, the conductive layer is patterned to allow additional electrodes. In some embodiments, the conductive layer has additional sensor chemistries deposited. In some embodiments, the conductive layer is unmodified. In some embodiments, the second conductive layer is adhered to the spacer material through thermal, mechanical, chemical or optical activation.

In some embodiments, a continuous manufacturing process is used and multiple depositions of active chemical components are deposited on a substrate material. In some embodiments, the conductive materials are patterned at different locations using the same or different patterning methods. In some embodiments, the conductive materials are assembled by mechanical lamination in combination with thermal, chemical or optical activation.

### Further Embodiments

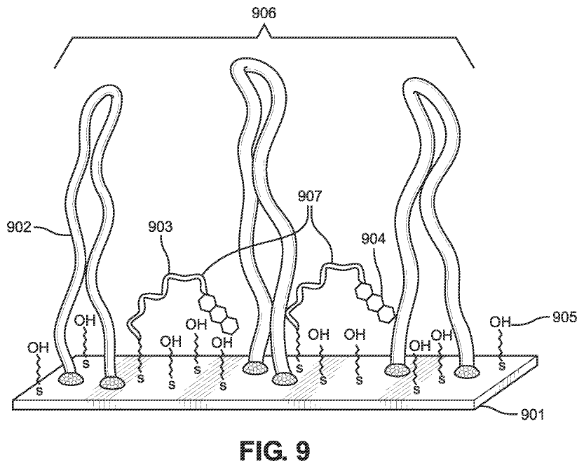

Double interpenetrating brush (i.e., the microRNA platform): As shown in FIG. 9, in some embodiments, the redox reporter (904) is not tethered directly to the brush (902) but is bound to a second end-grafted macromolecule (e.g., RNA, DNA, peptide) (903) which is bound directly to the electrode (901) forming a second, smaller brush (907) embedded within the larger active polymer brush (906) as shown in FIG. 9. The analyte binding is to the smaller brush (907) or to elements contained in the smaller brush.

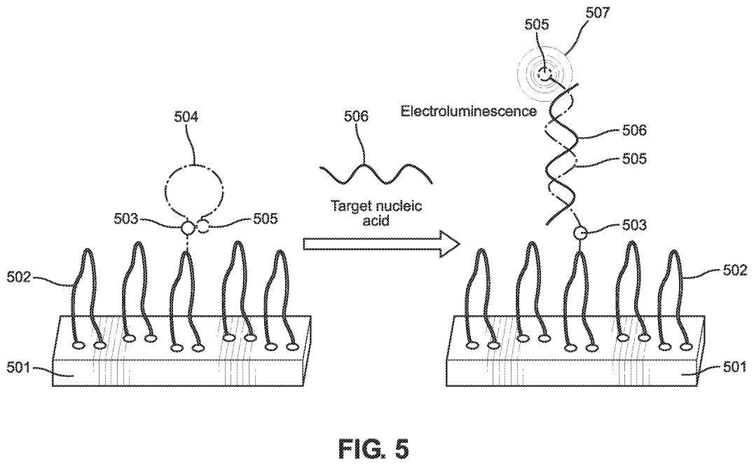

Electrochemiluminescence modified platform: As shown in FIG. 5, in some embodiments, the active polymer brush is adapted to perform electrochemiluminescence. For example, the second smaller brush (e.g., aptamer or single nucleic acid strand) (504) is modified with a redox reporter which is an electrochemiluminescence probe (505) and an electron acceptor which can act to quench the luminescence (503). Upon oxidation/reduction of this probe, a chromophore/luminophorc/fluorophore becomes activated causing the emission (or adsorption) of light (507) which can be detected and quantified. This only occurs in the presence of the analyte (506), which separates the acceptor (503) and the probe (505) allowing emission. The intensity of the light (or adsorption) is then used to quantify the amount of bound analytic.

Multiplexed arrays: In some embodiments, a multiplexed array consisting of recognition elements (i.e., multiple lectins) conjugated to the active polymer brush are used. Creating the array is achieved by using a series of individual, parallel arrayed sensors or by grafting multiple biorecognition elements modified with unique redox reporters (having unique oxidation and/or reduction potentials) to a single brush on a single sensor platform. Analysis of the signal intensity shifts coming from each of the individual biorecognition systems can thus be used to map the analyte binding affinity and create a library of analyte ‘fingerprints.’ For example, Tn derived from different cancer types have different affinities to different types of lectins due to variations in their glycome. This affinity variation is harnessed as a diagnostic tool that can both detect the presence of Tn and provide information about what types of cancer cells produced the Tn detected. This is done using a multiplexed array consisting of multiple lectins conjugated to the lubricin mucin domain.

Integration of binding domains within the polymer brush protein amino acid sequence: In some embodiments, the gene used in the recombinant synthesis of the protein is modified to introduce antibody, lectin, or other molecular binding domains into the protein. For example, the gene used in the recombinant synthesis of the lubricin molecule is modified to introduce antibody, lectin, or other molecular binding domains into the lubricin mucin domain region. Likewise, cystine or other functional amino acids can likewise be integrated into the mucin domain to allow direct functionalization of the lubricin with redox reporter species.

### Advantages

The following advantages (and embodiments throughout the application) can refer to a lubricin-tethered redox reporter biosensor (LTB) platform. However, other synthetic polymers, proteins, DNA or RNA sequences can be used in place of lubricin to provide the same advantages. Likewise other reporters can be used in place of a redox reporter.

The lubricin molecule performs several vital functions in the sensor mechanism which greatly simplifies the interfacial assembly and makes the sensor more amenable to mass production processing. First, the lubricin molecules serve as the anchor for the biorecognition element and redox reporter either as a single conjugated unit or separately. Second, the natural vibrational frequency of the lubricin molecules within the assembled lubricin brush structure provides the source of harmonic molecular dynamic motion central to the transduction of a binding event into a detectible change in electrical signal. Third, the lubricin brush functions as an anti-fouling agent which prevents non-specific adsorption of molecules/cells that electrically passivate the electrode or otherwise interfere with the electrochemical processes. The use of a single element that performs multiple vital functions in the sensor which traditionally has been performed by several, singular functional elements, simplifies the sensor design and reduces the number of required fabrication steps.

By inhibiting electrode fouling, it is possible to obtain improved electrochemistry by a combination of greater current/signal sensitivity and lower ‘noise’ levels which lead to improved signal fidelity and greatly lowered limits of detection when measurements are performed in complex, highly fouling media; e.g., unprocessed blood, saliva, tears, urine, and the like. This improvement in electrochemical signal quality is particularly pronounced for sensors in which slow analyte binding kinetics to the biorecognition element require long exposure/acquisition times.

The very large size and close packed brush structure of the lubricin molecules greatly slows down the molecular vibration frequencies/dynamics which likewise leads to improved signal intensities and lower detection limits due to improved electrochemistry. By comparison, end-tethered single DNA strands or aptamer/protein systems utilizing dynamic folding instabilities (e.g., E-DNA sensors and aptamer sensors) naturally vibrate at frequencies too fast to accurately ‘sample’ via square-wave voltammetry. To correct for these naturally high dynamic frequencies, it is necessary further modify the remaining electrode surface area with a ‘blocking’ layer consisting of a short chain thiol (or similar molecular monolayer) that partially passivates the electrode and artificially retards electron transfer rates (via reduced transfer efficiencies). The use of lubricin with its slower dynamic motion combined with the greater average distance of the redox probe from the electrode surface (which is another way of reducing charge transfer efficiency) eliminates the need to further block the electrode surface and simplifies the device fabrication.

Fabrication of the lubricin tethered biosensor does not involve chemical/covalent attachment of any chemical or biological moieties to the electrode surface. Instead, the sensor's vital interfacial structures self-assemble onto and adhere to electrode surfaces via physical interaction forces. Because self-assembly is an entropically driven process and does not rely on an enthalpically limited chemical reaction (whose reaction rate is thus limited by an activation energy barrier), it is possible to greatly accelerate the self-assembly of the lubricin interfacial structures through physical processes; e.g., the concentration of molecular species as a thin liquid film evaporates.

Because fabrication of the active polymer brush biosensor when using lubricin does not require chemical/covalent attachments, lubricin tethered biosensor sensors can be fabricated using any electrode material which lubricin adheres to. Electrode materials that can be used successfully with lubricin brushes include, but are not limited to, gold, silver, titanium, platinum, carbon (including carbon black, glassy carbon, graphene, graphene oxide, reduced graphene oxide, highly ordered pyrolytic graphite (HOPG), carbon nanotubes, and conductive diamond), conductive polymers (polypyrrole, PEDOT, polythiophene and functionalised version of such), and the like. The lubricin tethered biosensor is the only form of surface tethered redox reporter sensor that does not require any special chemistry to fabricate. Because many electrode materials exhibit higher impedance and higher electron transfer efficiencies compared with the gold (e.g., carbon electrodes), it is possible to obtain improved sensor performance by fabricating lubricin tethered biosensors on these alternative electrodes.

The Tn antigen version of the lubricin tethered biosensor is the only form of the surface tethered redox reporter sensor that utilizes lectin biorecognition elements.

Because the signal transduction mechanism depends upon the natural vibrational modes of lubricin molecules within the brush structure, it is the only form of the surface tethered redox reporter sensor that does not require engineered folding instabilities, DNA/RNA conjugation, or externally applied dynamic motion in order to generate dynamic harmonic motions. The sensor design is thus innately modular with analyte recognition elements interchangeable without significant change in the device manufacture or operation protocols. A sensor can thus be constructed using any recognition element that can both attached to the active brush and also bind an analyte.

The sweeps required to use the sensor can be much slower than are typically used (5-10 or 25 Hz range and even lower sometimes) due to the greater sensitivity of the sensor. Typical sweep in square wave pulse tuned to the system are about (60 Hz) (200, 300, 600 Hz), which reduces the cost of the electronics required for the measurement and reduces noise arising from fast measurements.

### EXAMPLES

### Example 1

A redox-reporter-modified protein site specifically tethered to a disposable electrode via a flexible, lubricin monolayer linker was made and tested for direct analysis of tumor antigens in a finger-prick volume of unprocessed whole blood.

Briefly, using reduced graphene oxide (rGO) screen-printed electrodes as a substrate functionalized with lubricin and redox-labelled peanut agglutinin (PNA), it was demonstrated that it is possible to detect Tn antigen levels directly in whole blood with high levels of selectivity and sensitivity and with a broad dynamic range. To convert the LUB-PNA into a single-step electrochemical sensor, firstly the single cysteine side chain of PNA was conjugated with a maleimide-functionalized methylene blue (MB-PNA). PNA is a plant-based lectin, which presents in its carbohydrate binding site a refined complementarity that allows interaction with their carbohydrate binding target, in this case the T and Tn antigens (22,23). This results in exceptional high specificity in these interactions. To create sensors, the MB-PNA was conjugated with the lubricin, which forms the active polymer brush providing three functions 1) to act as an antifouling agent and 2) to anchor the MB-PNA to the electrode surface (we refer to this as rGO/LUB/MB-PNA) and 3) to provide the molecular dynamics which enable the large dynamic range and high sensitivity of the sensors.

### Example 2

This Example provides methods of experiments performed using the sensor of Example 1.

PNA-methylene blue conjugation: Methylene blue conjugation to lectin from Arachis hypogaear (Sigma Alrdich) was performed under gentle mixing overnight in PBS, pH 7.4 (Sigma-Aldrich, Castle Hill, Australia), using ATTO-MB2 maleimide derivative methylene blue (Sigma-Aldrich, Castle Hill, Australia). 20 μL of ATTO-MB2 (6 μM) was added to 900 μL of PNA (0.8 μM) and incubated overnight. Peanut agglutinin presents a single cysteine site making it a good protein for functionalization with molecules containing thiol groups. Excess of methylene blue was removed by dialysis against 20 mM sodium phosphate, 130 mM NaCl, pH 7.0.

Surface modification of disposable rGO electrodes with LUB/PNA-MB: 20 L of the above solution was mixed with 20 μL of recombinant lubricin 100 μg mL⁻¹ (Lubris Biopharma, Boston, USA) and incubated while gentle mixing for two hours at room temperature in the dark. Next a 10 μL of the resulting solution was drop-casted into the disposable rGO electrodes (Orion High Technologies S.L., Madrid, Spain) and incubated for one hour. After the incubation the modified electrodes were rinsed thoroughly with Milli-Q water (>18 M (2 cm).

Tn antigen hybridization protocol: Tn antigen solutions with different concentrations, ranging between 54 PM and 1.4 μM, were prepared by serial dilution of a stock solution of target Tn antigen (Sigma-Aldrich, Castle Hill, Australia) using PBS. The prepared solutions were spiked to control PBS or human whole blood as a matrix. Whole blood samples used in this experiment were commercially available products (anticoagulated with K2 EDTA) acquired from Innovative Research (Novi, USA)) from a healthy human donor and from a donor diagnosed with breast cancer.

Electrochemical experiments: Electrochemical measurements were performed at room temperature using a CHI potentiostat (CHI instruments, model no. 600-60D). A 50 μL droplet of PBS or whole blood sample was added to the electrode surface. The disposable rGO electrodes from Orion High Technologies S.L. are comprised of nanostructured carbon (4 mm diameter) functionalized with reduced graphene oxide as working electrode, carbon as counter electrode, and Ag|AgCl as reference electrode.

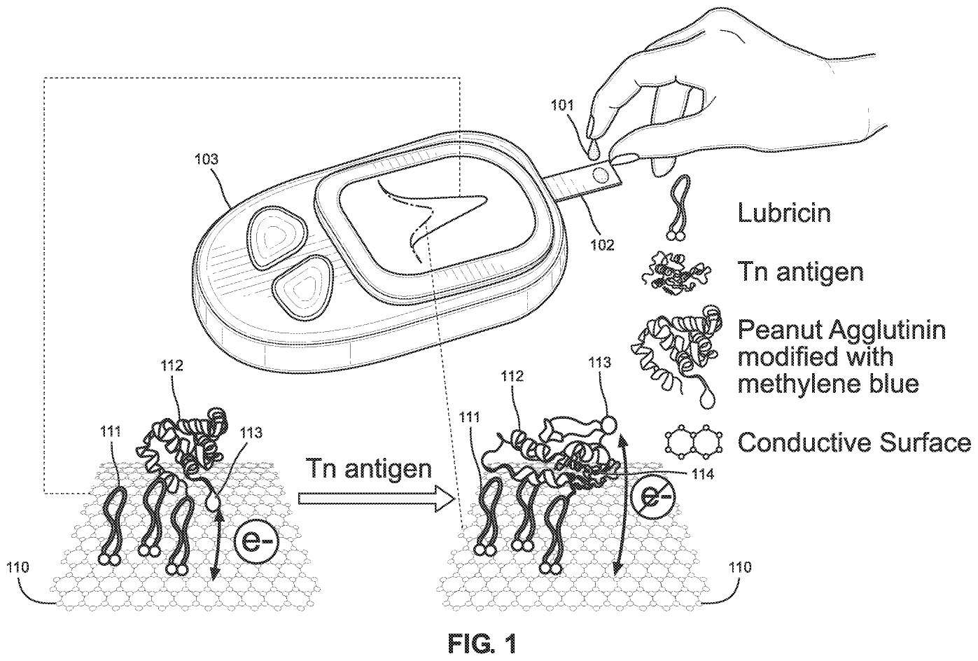

In the absence of Tn antigen target, the redox reporter was relatively free to collide with the electrode surface, generating a high faradaic current at the redox potential predicted for methylene blue when the electrode is interrogated using square wave voltammetry (see FIG. 1). This peak was then smaller in magnitude in the presence of the Tn antigen.

Using the new protein-based electrochemical sensor, direct analysis of Tn antigen in a finger-prick volume of whole blood with high sensitivity over six orders magnitude of concentration (54 PM-1.35 μM) was demonstrated.

### Example 3

The electrochemical behavior of the sensor of Example 1 was tested using methods of Example 2. In a previous study it was demonstrated by using atomic force microscopy to evaluate the adhesion energy, adhesion force range and long-range modulus, that lubricin itself forms an organized self-assembled monolayer on rGO electrodes (15). Here, before assembling all the surface components on rGO, quartz crystal microbalance (QCM) was used to investigate the biding between LUB and MB-PNA.

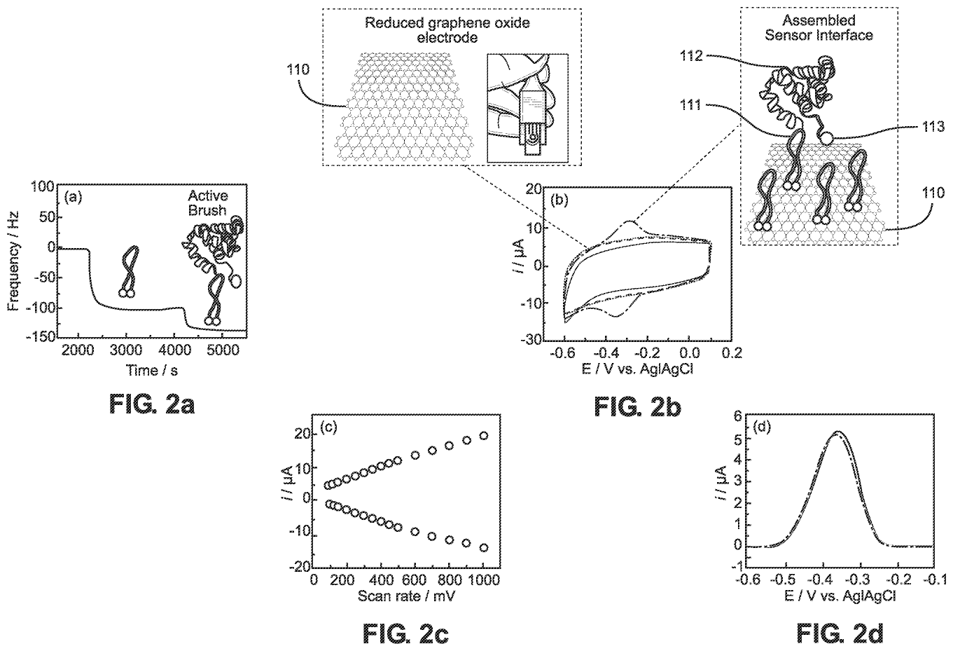

FIG. 2a shows a representative QCM measurement (performed on a gold-coated QCM sensor surface in phosphate buffer saline solution, PBS) displaying how the measured frequency changes in time during the measurement. In a first instance, the QCM crystal was equilibrated with PBS solution at a constant flow rate of 300 μL/min until a stable frequency baseline was achieved. In a second stage (approximately 1,800 s), 100 μL of lubricin protein solution (100 μg mL⁻¹) was flowed into the QCM flow cell. After all the lubricin protein solution had been flowed into the cell with a flow rate of 100 μL min-1, the flow was stopped for an incubation period of 30 minutes. Once the incubation period was finalized, the surface was washed by flowing fresh PBS solution in the flow cell at a constant rate of 300 μL min⁻¹ to remove lubricin excess until a new stable baseline frequency was obtained. In a third stage, 100 μL of 0.1mgmL⁻¹ methylene blue conjugated PNA solution was flowed into the QCM flow cell at a flow rate of 100 μL min⁻¹ and incubated for 15 minutes. Finally, after the incubation time, the cell was once again rinsed with PBS solution to remove any unbound or weekly adsorbed MB-PNA. The first shift in frequency after the introduction of lubricin to the QCM cell suggested that the lubricin immobilizes to the gold crystal (13,14), and the shift after MB-PNA introduction suggested the successful immobilization of MB-PNA to the lubricin monolayer.

The success of the surface modification was further confirmed using cyclic voltammetry. FIG. 2b show the cyclic voltammograms for the bare rGO electrode (solid line), the rGO modified with lubricin only (dashed line), the rGO modified with lubricin and PNA (without the redox reporter) (dotted line), and rGO electrode modified with lubricin and MB-PNA (dot-dashed line) recorded in PBS solution using a scan rate of 100 mV s⁻¹. As expected, due to the absence of electrochemical active species, no redox peaks were observed. When the rGO electrode was modified with the solution containing lubricin and MB-PNA, symmetric oxidation and reduction peaks corresponding to the electrochemistry of methylene blue redox labelled at potential of approximately −350 mV with peak-to-peak separation of 60 mV was observed. This suggested that the majority of methylene blue redox labels bonded to PNA were capable of accessing the electrode surface within the time frame of the voltammogram at this scan rate.

As observed in FIG. 2c, the anodic and cathodic currents were proportional to the scan rates in the range from 100 to 1000 mV s⁻¹. This characteristic is consistent with the voltametric signal originating from a surface-confined species exhibiting ideal Nernstian behaviour (24).

FIG. 2d shows the representative electrochemical behavior of the rGO/LUB/MB-PNA sensor using square-wave voltammetry as excitation technique and PBS as supporting electrolyte. Similar to the data obtained using cyclic voltammetry, the redox current peak for methylene blue was located in approximately −350 mV (solid line). In order to evaluate the shelf-life of the prepared rGO/LUB/MB-PNA sensor, the same surface as presented in FIG. 2d (solid line) was stored inside a desiccator at room temperature for six months and a second measurement was then performed in PBS (dot-dashed line). In these studies, negligible changes were observed, which shows that the proposed surface presents extended shelf-life without the need to impose strict temperature conditions.

### Example 4

The sensor of Example 1 demonstrated ultrasensitive quantification of Tn antigen in experiments using methods of Example 2.

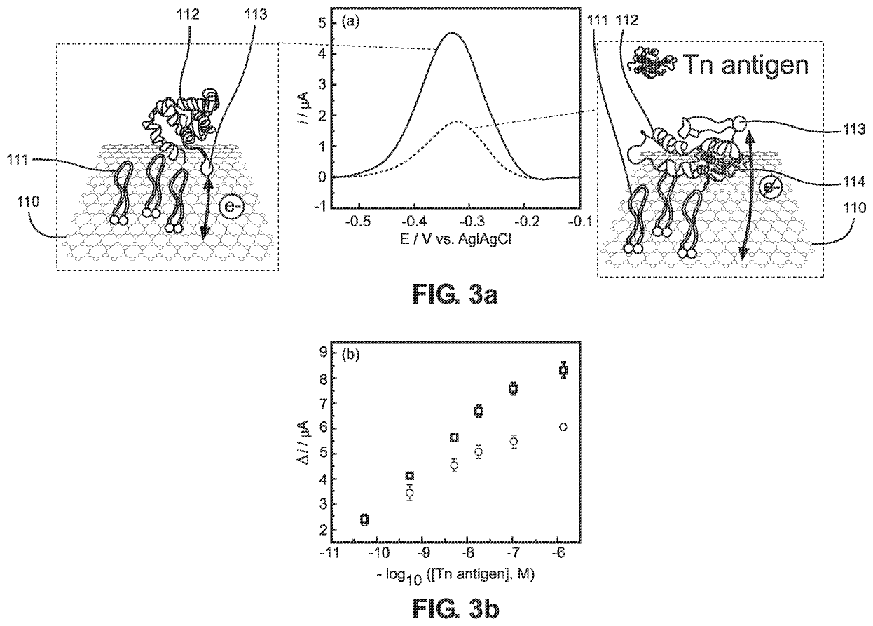

FIG. 3a shows representative square wave voltammograms recorded in whole blood samples from a healthy donor for the rGO/LUB/MB-PNA, before (solid line) and after (dashed line) spiking 5.15×10⁻⁹ M Tn antigen target. Background peak currents were subtracted from the square-wave voltammograms to give the presented results. As the theoretical quantity of redox reporters does not change by the binding to the target Tn antigen, suppression in the current subsequent to binding suggests a mechanism by which recognition of the specific antigen alters the electronic communication between redox reporter and electrode surface. The average 4.54±0.25% reduction in the magnitude of methylene blue electrochemical current observed after specific binding with this particular concentration of Tn antigen, infers that a decrease in the current is a systematic effect triggered by interaction of the analyte with the recognition element, modifying the methylene blue interactions with the electrode surface.

To evaluate the limit of detection and dynamic range of the sensor, different concentrations of Tn antigen were prepared using serial dilutions of Tn antigen either in PBS solution (squares) or in whole human blood (circles) as the two matrixes (FIG. 3b). The whole human blood utilized in these experiments was obtained from a healthy human donor. A measurable change in the current, compared to that before the specific binding, was observed after 2 minutes exposure of the sensor to Tn antigen solution within the concentration range of 54.0 pM to 1.35 μM. The lowest detectable concentration for Tn antigen was 54.0 pM, which is comparable to the laboratory-based methodology high-performance liquid chromatography (HPLC) (25) or immunofluorescence (26), without the need for sample purification and long analysis time (˜4 hours). It is important to note this is the first electrochemical assay for Tn antigen detection capable of functioning in unprocessed blood samples in a clinically reasonable time-frame (˜5 minutes).

### Example 5

The sensor of Example 1 was used to test whole blood patient samples using methods of Example 2.

Glycosylation has been recognized to be vital in a range of different cancer cell processes, as for example tumor angiogenesis, immune response modulation, interaction with tumor microenvironment and metastasis formation (27). Hence, alterations of the cellular surface glycans have critical structural and functional consequences for cancer cells, influencing the progression of the disease and impacting prognosis of patients (27). It has been shown that extracellular vesicles present key functions in the horizontal transfer of material from donor to recipient cells, by this means acting as important intercellular communication tools in physiological and pathological settings (28-30). Moreover, previous studies have demonstrated that extracellular vesicles are crucial in the modulation and establishment of the pre-metastatic niche, responsible for cancer progression and metastasis (31-33). Consequently, beyond the potential to differently interplay with other cells within the tumor microenvironment as well as at distant sites (26), extracellular vesicles exhibiting dissimilar ‘glycan coats’ are of interest as potential biomarkers for cancer diagnosis and screening.

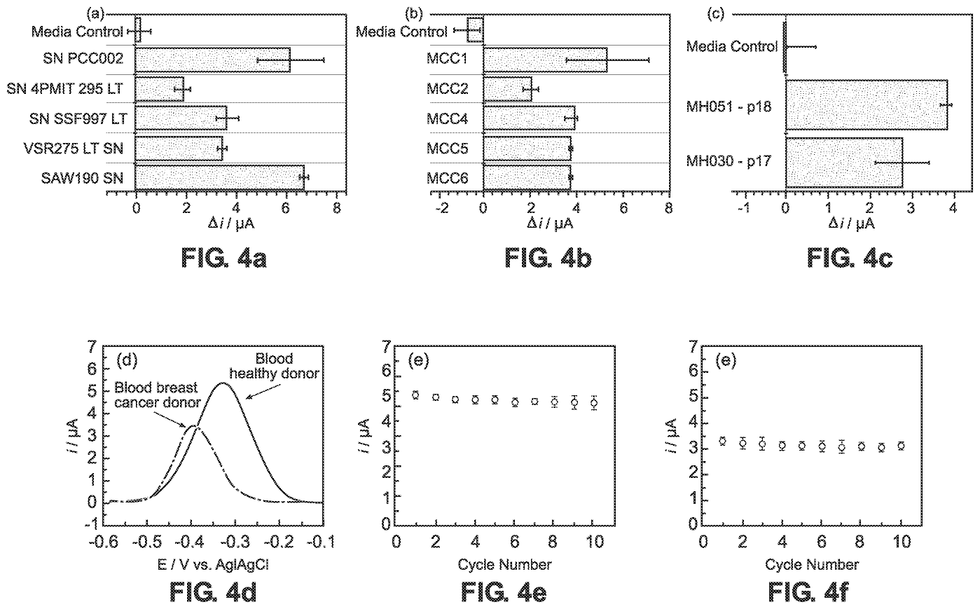

The sensor of Example 1 was tested by analyzing Tn antigen levels in a pool of extracellular vesicles released by the cells into culture medium (the analysis was performed in an aliquot of supernatant of each cell line). Three different cancer cell lines from different cancer models were investigated, liver metastasis (FIG. 4a), Merkel-cell carcinoma (FIG. 4b) and pancreatic cancer (FIG. 4c). For these experiments, the voltametric currents were recorded twice, one time in PBS solution only and subsequently in each cell culture media. Hence, the difference in binding induced current change could be calculated. For all cancer cell lines, the media control presented negligible current change suggesting that electron transfer for the methylene blue in the cell media alone is comparable to the PBS solution. However, for all three different cancer cell models studied, it was possible to observe that Tn antigen binding induces the current change. Given that the sensor was able to detect such marked variations in the levels of Tn antigen expression suggests that it has potential as a precise and selective approach for analyzing variations in the expression levels of Tn antigen in the presence of a pool of other tumor associated antigens.

As shown in FIG. 4d, to further examine the capability of the sensor for direct detection of Tn antigen in clinical samples, the levels of Tn antigen were quantified (frequency 25 Hz, pulse amplitude 20 mV) in whole blood sample from a single donor with breast cancer (solid line) and compared against analysis in whole blood sample from a healthy donor (dot-dashed line). Background peaks were subtracted from the square-wave voltammograms to give the presented results. As observed, the voltametric peak current obtained for the rGO/LUB/MB-PNA when challenged in whole blood from a healthy donor was approximately 5.3 μA, while when the sensor was interrogated in the blood sample from a cancer donor, the current was approximately 3.13 μA. Additionally, the methylene blue peak potential shifted to more negative potential when interrogated in the blood sample from the cancer donor. A decrease in the current level together with a shift in the peak potential suggested that a majority of the methylene blue-peanut agglutin had been specifically bonded to Tn antigen in the blood sample and that the remaining current originated mainly from the free methylene blue-peanut agglutin probes (i.e., not bound to Tn), which can still collide with the electrode surface, albeit at a reduced rate.

FIGS. 4e and 4f presents the pattern of change in the square wave current through ten cycles (frequency 25 Hz, pulse amplitude 25 mV), after rGO/LUB/MB-PNA sensor was exposed to whole blood from the healthy donor (FIG. 4c) and blood from the donor with breast cancer (FIG. 4f). In both cases, the current remained unchanged with electrical pulsing for 10 cycles of square-wave voltammetry, suggesting the current response was stable with further electrical pulsing.

### Example 6

### FABPI to PFAS

The methods of manufacture and measurement for the sensor of Example 1 and 2 were used.

The analyte recognition of the sensor of Example 1 was expanded to the detection of perfluorooctanoic acid (PFOA), which is classified as a perfluorinated alkyl substance (PFAS). Contamination of water by PFAS is an emerging global issue due to their alleged toxicity and capability to bioaccumulate. PFAS are persistent in the environment and can be found at high concentrations in polluted areas for years, even after their production or use was stopped. Therefore, there is a pressing need to develop technologies able to monitor levels of PFAS on-site.

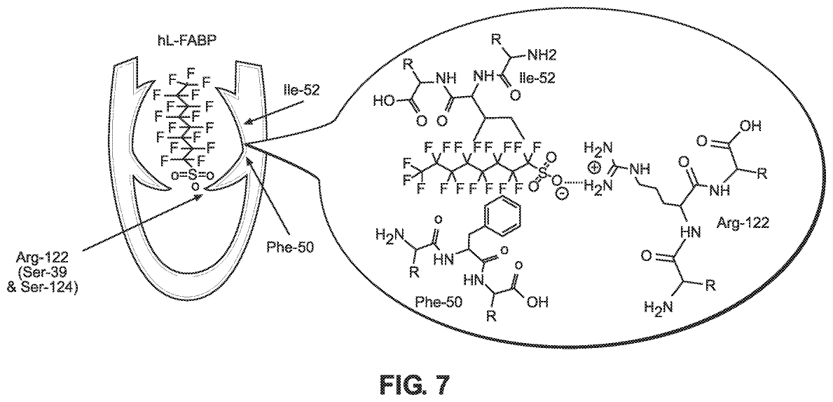

Fatty-acid binding protein from human liver (FABP1) was used as a recognition element to specifically detect PFOA. The specific affinity between PFOA and FABPI can be described by a combination of polar and hydrophobic interactions. FIG. 7 shows the proposed mechanism of the binding between PFAS and human liver fatty acid-binding protein (figure reproduced from reference 35 Environ. Sci. Technol. 2020, 54, 5676-5686). The polar interaction between PFOA and FABPI protein relies on the negative charge of acidic head groups to maintain ionic and hydrogen bonds with Arg-122, Ser-39, and Ser-124. Additionally, hydrophobic interactions are formed between alkyl chains partition of PFOA and Phe-50 and Ile-52 in the protein (FIG. 7).

Fatty-acid binding protein from human liver (FABP1)-methylene blue conjugation: Methylene blue conjugation to Fatty-acid binding protein from human liver (Sigma Alrdich) was performed under gentle mixing overnight in PBS, pH 7.4 (Sigma-Aldrich, Castle Hill, Australia), using ATTO-MB2 maleimide derivative methylene blue (Sigma-Aldrich, Castle Hill, Australia). 25 μL of ATTO-MB2 (6 μM) was added to 900 μL of FABP1 (0.8 μM) and incubated overnight. Similar to peanut agglutinin, FABPI also presents a single cysteine site making it a good protein for functionalization with molecules containing thiol groups. Excess of methylene blue was removed by dialysis against 20 mM sodium phosphate, 130 mM NaCl, pH 7.0. The resulting conjugated is here referred as FABP1-MB.

Surface modification of disposable reduced graphene oxide (rGO) electrodes with FABP1-MB. 20 μL of LUB (Lubris Biopharma, Boston, USA) was drop-casted directly onto the disposable rGO electrodes (Orion High Technologies S.L., Madrid, Spain) and incubated for 15 minutes. After the incubation, the modified electrodes were rinsed thoroughly with Milli-Q water (>18 MΩ cm). In the second step, 20 μL of FABPI-MB was drop-casted on the electrodes and incubated for 1 hour and 30 minutes. After the incubation, the modified electrodes were rinsed thoroughly with Milli-Q water (>18 MΩ cm) and the electrode was ready to use.

PFOA hybridization protocol: PFOA solutions with different concentrations, ranging between 96 nM and 966 μM, were prepared by serial dilution of a stock solution of target Tn antigen (Sigma-Aldrich, Castle Hill, Australia) using PBS. The prepared solutions were spiked to control phosphate buffer saline as a supporting electrolyte. The solutions with different PFOA concentrations were directly spiked on the electrode surface for analysis.

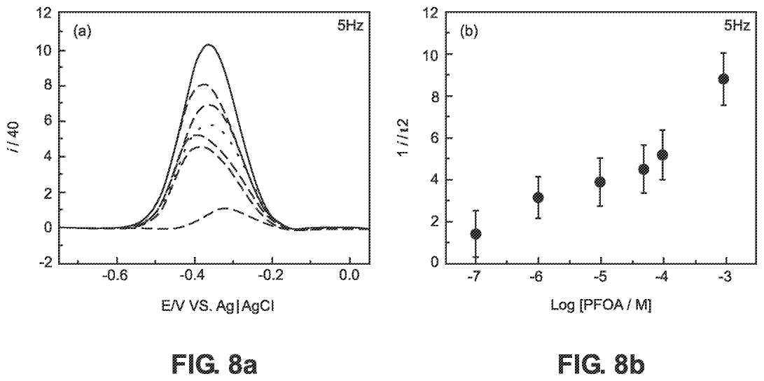

Electrochemical experiments: Electrochemical measurements were performed at room temperature using a CHI potentiostat (CHI instruments, model no. 600-60D). The disposable rGO electrodes from Orion High Technologies S.L. are comprised of nanostructured carbon (4 mm diameter) functionalized with reduced graphene oxide as a working electrode, carbon as a counter electrode, and Ag|AgCl as a reference electrode. To evaluate the limit of detection and dynamic range of the sensor, different concentrations of PFOA were prepared using serial dilutions of PFOA in phosphate buffer saline as supporting electrolyte (FIG. 8). As shown in FIG. 8a, as observed in the square-wave voltammogram, a measurable change in the current, compared to that before the specific binding (solid line), was observed after the exposure of the sensor to PFOA solution within the concentration range of 96 nM to 966 μM (dashed lines). Experiments were recorded using 5 Hz. As the concentration of spiked PFOA increased, the electrochemical signal decreased proportionally. FIG. 8b shows the calibration curve for these studies, where the lowest detectable concentration for PFOA was 96 nM.

### Example 7

### Nucleic Acid LUB Embedded Sensor

This Example provides methods of experiments performed for the generation of an electrochemical sensor with an electrode tethered reporter embedded in an active polymer brush, redox labelled aptamer embedded in lubricin brush.

These experiments were performed with the commercial polycrystalline gold electrode (CH Instruments, Austin USA) for the electrochemical detection of microRNA-375 (miRNA-375). Each electrode surface was first polished with wet alumina slurries with particle sizes of 1.0, 0.3, and 0.05 μm on polishing cloths (Thermo Fisher Scientific, Sydney). Subsequently, the electrodes were rinsed with Milli-Q water and electrochemically cleaned in 0.05 M H₂SO₄ by cycling in the potential range from −0.1 to 1.5 V vs. Ag|AgCl (KCl 3 M) at 100 mV s⁻¹ for 25 scans. After the surfaces were rinsed with Milli-Q water and ethanol.

Surface preparation: The clean electrodes were used to tether the nucleic acid capture probes for miRNA-375. Immobilization of DNA was carried out by incubating the clean electrodes for 2 hours in 10 μL of 1 μM DNA probes solution containing the redox label methylene blue in phosphate buffer saline, pH 7.4, at room temperature. Afterward, the electrodes were thoroughly rinsed with phosphate buffer saline to remove any excess reagents or any weakly attached nucleic acid strands. In a second step, 20 μL of recombinant LUB 100 μg mL⁻¹ prepared in phosphate buffer saline (Lubris Biopharma, Boston, USA) was drop-casted onto the electrode surface and incubated for half hour. Once again, the electrodes were thoroughly rinsed with phosphate buffer saline to remove any excess reagents. To finalize the electrode preparation, 20 μL of a 2 mM mercaptohexan-1-nol (MCH) solution in ethanol was drop-cast on the electrode surface and incubated for 30 minutes to produce the surface represented schematically in FIG. 9, followed by rinsing with PBS.

miRNA-375 hybridization protocol: Hybridization of target miRNA-375 was performed at room temperature by drop-casting the desired concentration solutions of the target miRNA-375 prepared in phosphate buffer saline or unprocessed human blood on the prepared electrode surface and incubating for fifteen minutes prior to the electrochemical measurement.

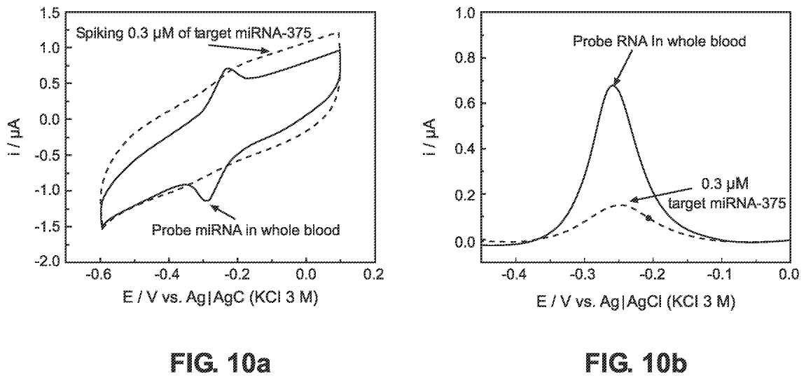

Electrochemical experiments: Electrochemical measurements were performed at room temperature using a CHI potentiostat (CHI instruments, model no. 600-60D). The experiments were carried out in a conventional three-electrode cell, with an Ag|AgCl (KCl 3 M) electrode as reference electrode and a platinum electrode as the counter electrode. FIGS. 10a and 10b shows a cyclic voltammogram and square-wave voltammogram respectively, recorded in a whole blood sample from a healthy donor (solid line) and whole blood spiked with 0.3 μM miRNA-375 (dashed line). As observed a very well define redox peak is obtained for methylene blue in the whole blood sample without the miRNA-375. Upon the spiking of miRNA-375 the redox peak current on the cyclic voltammogram completely disappears as the electrochemical signal is switched off. Similar behavior is observed on the square-wave voltammogram.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES CITED

The disclosure of each of the references below is hereby incorporated by reference in its entirety.
ADDIN EN.REFLIST 1 Barfidokht, A. & Gooding, J. J. Approaches Toward Allowing Electroanalytical Devices to be Used in Biological Fluids. Electroanal 26, 1182-1196, doi: 10.1002/elan.201400097 (2014).
2 Lisi, F., Peterson, J. R. & Gooding, J. J. The application of personal glucose meters as universal point-of-care diagnostic tools. Biosensors and Bioelectronics 148, 111835, doi: https://do 019.111838 (2020).
3 Gooding, J. J. Finally, a simple solution to biofouling. Nature Nanotechnology 14, 1089-1090, doi: 10.1038/s41565-019-0573-0 (2019).
4 Sabaté del Río, J., Henry, O. Y. F., Jolly, P. & Ingber, D. E. An antifouling coating that enables affinity-based electrochemical biosensing in complex biological fluids. Nature Nanotechnology 14, 1143-1149, doi: 10.1038/s41565-019-0566-z (2019).
5 Lubin, A. A. & Plaxco, K. W. Folding-Based Electrochemical Biosensors: The Case for Responsive Nucleic Acid Architectures. Accounts of Chemical Research 43, 496-505, doi: 10.1021/ar900165x (2010).
6 Katz, E. & Willner, I. Probing Biomolecular Interactions at Conductive and Semiconductive Surfaces by Impedance Spectroscopy: Routes to Impedimetric Immunosensors, DNA-Sensors, and Enzyme Biosensors. Electroanal 15, 913-947, doi: 10.1002/elan.200390114 (2003).
7 Baker, B. R. et al. An Electronic, Aptamer-Based Small-Molecule Sensor for the Rapid, Label-Free Detection of Cocaine in Adulterated Samples and Biological Fluids. J Am Chem Soc 128, 3138-3139, doi: 10.1021/ja056957p (2006).
8 Fan, C., Plaxco, K. W. & Heeger, A. J. Electrochemical interrogation of conformational changes as a reagentless method for the sequence-specific detection of DNA. Proceedings of the National Academy of Sciences 100, 9134-9137, doi: 10.1073/pnas. 1633515100 (2003).
9 Xiao, Y., Lubin, A. A., Heeger, A. J. & Plaxco, K. W. Label-Free Electronic Detection of Thrombin in Blood Serum by Using an Aptamer-Based Sensor. Angewandte Chemie International Edition 44, 5456-5459, doi: 10.1002/anie.200500989 (2005).
10 Dauphin-Ducharme, P. et al. Electrochemical Aptamer-Based Sensors for Improved Therapeutic Drug Monitoring and High-Precision, Feedback-Controlled Drug Delivery. ACS Sensors 4, 2832-2837, doi: 10.1021/acssensors.9b01616 (2019).
11 Parolo, C. et al. E-DNA scaffold sensors and the reagentless, single-step, measurement of HIV-diagnostic antibodies in human serum. Microsystems & Nanoengineering 6, 13, doi: 10.1038/s41378-019-0119-5 (2020).
12 Greene, G. W. et al. Lubricin: A versatile, biological anti-adhesive with properties comparable to polyethylene glycol. Biomaterials 53, 127-136, doi: 10.1016/j.biomaterials.2015.02.086 (2015).
13 Greene, G. W. et al. Lubricin Antiadhesive Coatings Exhibit Size-Selective Transport Properties that Inhibit Biofouling of Electrode Surfaces with Minimal Loss in Electrochemical Activity. Adv Mater Interfaces 5, doi: ARTN 1701296, 10.1002/admi.201701296 (2018).
14 Han, M. Y. et al. Adhesion and Self-Assembly of Lubricin (PRG4) Brush Layers on Different Substrate Surfaces. Langmuir 35, 15834-15848, doi: 10.1021/acs.langmuir.9b01809 (2019).
15 Russo, M. J. et al. Lubricin (PRG4) reduces fouling susceptibility and improves sensitivity of carbon-based electrodes. Electrochim Acta 333, doi: ARTN 135574, 10.1016/j.electacta.2019.135574 (2020).
16 Silva, S. M., Quigley, A. F., Kapsa, R. M. I., Greene, G. W. & Moulton, S. E. Lubricin on Platinum Electrodes: A Low-Impedance Protein-Resistant Surface Towards Biomedical Implantation. Chemelectrochem 6, 1939-1943, doi: 10.1002/celc.201900237 (2019).
17 Nativi, C., Papi, F. & Roelens, S. Tn antigen analogues: the synthetic way to “upgrade” an attracting tumor associated carbohydrate antigen (TACA). Chem Commun 55, 7729-7736, doi: 10.1039/c9cc02920f (2019).
18 Ju, T. Z., Otto, V. I. & Cummings, R. D. The Tn Antigen-Structural Simplicity and Biological Complexity. Angew Chem Int Edit 50, 1770-1791, doi: 10.1002/anie.201002313 (2011).
19 Julien, S., Videira, P. A. & Delannoy, P. Sialyl-Tn in Cancer: (How) Did We Miss the Target? Biomolecules 2, 435-466, doi: 10.3390/biom2040435 (2012).
20 Hollingsworth, M. A. & Swanson, B. J. Mucins in cancer: Protection and control of the cell surface. Nat Rev Cancer 4, 45-60, doi: 10.1038/nrc 1251 (2004).
21 Tavallaie, R. et al. Nucleic acid hybridization on an electrically reconfigurable network of gold-coated magnetic nanoparticles enables microRNA detection in blood. Nature Nanotechnology 13, 1066-1071, doi: 10.1038/s41565-018-0232-x (2018).
22 He, X.-P. et al. Epimeric Monosaccharide-Quinone Hybrids on Gold Electrodes toward the Electrochemical Probing of Specific Carbohydrate-Protein Recognitions. J Am Chem Soc 133, 3649-3657, doi: 10.1021/ja110478j (2011).
23 Poiroux, G., Barre, A., Van Damme, E. J. M., Benoist, H. & Rouge, P. Plant Lectins Targeting O-Glycans at the Cell Surface as Tools for Cancer Diagnosis, Prognosis and Therapy. International Journal of Molecular Sciences 18, 1232 (2017).
24 Bard, A. J. & Faulkner, L. R. Electrochemical methods: fundamentals and applications. 2nd edn, (Wiley, 2001).
25 Yamada, K., Hirabayashi, J. & Kakehi, K. Analysis of O-Glycans as 9-Fluorenylmethyl Derivatives and Its Application to the Studies on Glycan Array. Analytical Chemistry 85, 3325-3333, doi: 10.1021/ac303771q (2013).
26 Freitas, D. et al. Different isolation approaches lead to diverse glycosylated extracellular vesicle populations. Journal of Extracellular Vesicles 8, 1621131, doi: 10.1080/20013078.2019.1621131 (2019).
27 Pinho, S. S. & Reis, C. A. Glycosylation in cancer: mechanisms and clinical implications. Nat Rev Cancer 15, 540-555, doi: 10.1038/nrc3982 (2015).
28 van Niel, G., D'Angelo, G. & Raposo, G. Shedding light on the cell biology of extracellular vesicles. Nature Reviews Molecular Cell Biology 19, 213-228, doi: 10.1038/nrm.2017.125 (2018).
29 Rak, J. & Guha, A. Extracellular vesicles-vehicles that spread cancer genes. BioEssays 34, 489-497, doi: 10.1002/bies.201100169 (2012).
30 Rosa-Fernandes, L., Rocha, V. B., Carregari, V. C., Urbani, A. & Palmisano, G. A Perspective on Extracellular Vesicles Proteomics. Frontiers in Chemistry 5, doi: 10.3389/fchem.2017.00102 (2017).
31 Costa-Silva, B. et al. Pancreatic cancer exosomes initiate pre-metastatic niche formation in the liver. Nature Cell Biology 17, 816-826, doi: 10.1038/ncb3169 (2015).
32 Hoshino, A. et al. Tumor exosome integrins determine organotropic metastasis. Nature 527, 329-335, doi: 10.1038/nature15756 (2015).
33 Peinado, H. et al. Pre-metastatic niches: organ-specific homes for metastases. Nat Rev Cancer 17, 302-317, doi: 10.1038/nrc.2017.6 (2017).
34 Flowers, S A. Lane, C S. and Karlsson, N G. Deciphering Isomers with a Multiple Reaction Monitoring Method for the Complete Detectable O-Glycan Repertoire of the Candidate Therapeutic, Lubricin Analytical Chemistry 91 (15), 9819-9827, doi: 10.1021/acs.analchem.9b01485 (2019).
35 Yang. D. et al. Nontarget Screening of Per- and Polyfluoroalkyl Substances Binding to Human Liver Fatty Acid Binding Protein, Environmental Science & Technology 2020 54 (9). 5676-5686 doi: 10.1021/acs.est.0c00049

1. An electrochemical sensor for determining the presence of an analyte in a liquid sample, comprising:
a. an electrode having a surface;
b. an active polymer brush;
c. an analyte recognition element bound to the polymer brush; and
d. a signal transduction element comprising a reporter,
wherein the analyte recognition element specifically interacts with an analyte, when present, resulting in a detectable change in a charge transfer between the reporter and the electrode.

2. An electrochemical sensor for determining the presence of an analyte in a liquid sample, comprising:
a. an electrode;
b. a semi-permeable, antifouling polymer brush;
c. an analyte recognition element bound to the electrode and residing wholly within the polymer brush; and
d. a signal transduction element comprising a reporter,
wherein the analyte recognition element specifically interacts with an analyte, when present, resulting in a detectable change in a charge transfer between the reporter and the electrode.

3. The sensor of claim 1, wherein the electrode is carbon-based, metal-based, conductive metal oxide-based, or conductive polymer-based.

4. (canceled)

5. (canceled)

6. (canceled)

7. The sensor of claim 1, wherein the polymer brush has anti-fouling properties.

8. The sensor of claim 1, wherein the polymer brush is tethered to the electrode surface.

9. The sensor of claim 8, wherein the polymer brush is end-tethered to the electrode surface.

10. The sensor of claim 1, wherein the polymer brush is a telechelic polymer tethered at each end to the electrode surface.

11. The sensor of claim 1, wherein the polymer brush comprises a synthetic polymer or a biopolymer.

12. (canceled)

13. The sensor of claim 12, wherein the polymer brush comprises lubricin.

14. The sensor of claim 1, wherein the reporter is a redox reporter or an electro-chemiluminescent species.

15. (canceled)

16. The sensor of claim 1, wherein the analyte recognition element is a protein.

17. The sensor of claim 16, wherein the analyte recognition element is an antibody, a lectin, a nucleic acid, or an aptamer.

18. (canceled)

19. (canceled)

20. (canceled)

21. The sensor of claim 1, wherein the liquid sample is selected from blood, serum, saliva, urine, sweat, interstitial fluid, spinal fluid, cerebral fluid, tissue exudates, macerated tissue samples, cell solutions, intracellular compartments, water, food, groundwater, or other biological and environmental samples.

22. The sensor of claim 21, wherein the liquid sample is a complex liquid sample.

23. The sensor of claim 22, wherein the complex liquid sample requires minimal or no processing prior to use.

24. The sensor of claim 21, wherein the liquid sample is whole blood.

25. A sensor device comprising at least two electrodes each having an active surface that interact with the liquid sample, wherein a first electrode contains the sensor of claim 1 or 2 and a second electrode provides a current and reference potential.

26. The device of claim 25, wherein the first and second electrode active surfaces are opposing and separated by a non-conductive spacer that forms a channel into which the liquid sample can be introduced.

27. The device of claim 25, wherein the first and second electrode active surfaces are co-planar.

28. A sensor device comprising at least three electrodes in a combination of opposing and co-planar arrangement, the sensor device comprising:
a. at least two substrate materials coated with conductive material; wherein at least one of the two substrate materials coated with conductive material is patterned by printing, mechanical punching or laser scoring/ablation to isolate at least two separate electrodes thereon, wherein at least one electrode has active chemistry deposited on it to provide sensitivity and selectivity to an analyte of interest; and
b. a nonconductive spacer material used to separate the at least two substrate materials coated with conductive material and to define a fixed volume within the sensor device.
